(19) United States
(12) Patent Application Publication
(10) Pub. No.: US 2021/0356419 A1
(43) Pub. Date: Nov. 18, 2021

(54) HIGH DYNAMIC RANGE FAST CV SENSOR USING WIDE BANDGAP SILICON CARBIDE

(71) Applicant: University of South Florida, Tampa, FL (US)

(72) Inventors: Stephen Edward Saddow, Land O Lakes, FL (US); Christopher Leroy Frewin, Ann Arbor, MI (US)

(21) Appl. No.: 17/320,352

(22) Filed: May 14, 2021

Related U.S. Application Data
(60) Provisional application No. 62/704,562, filed on May 15, 2020.

(51) Int. Cl. G01N 27/30; G01N 27/48

(52) U.S. Cl. CPC G01N 27/308; G01N 27/48

(57) ABSTRACT

A fast scan cyclic voltammetry (CV) electrochemical voltammetry sensor comprises silicon carbide (SiC). The SiC may be single crystal SiC, and may be comprised within a SiC electrode. A system comprises a SiC electrode, and an applied voltage that is configured to apply voltage to the SiC electrode, wherein the voltage is swept within a range from a negative value to a positive value repeatedly and rapidly. The SiC electrode is configured to act as a biosensor in a CV process. The applied voltage is configured to be applied to the SiC electrode as a physiological species passes within a distance of the surface of the SiC electrode. A computing device may receive an output from the physiological species, and use the output in a biomedical application. The biomedical application may be a COVID-based application. The range may be −2V to +2.8V.

### CROSS-REFERENCE TO RELATED APPLICATIONS

This application claims the benefit of priority to U.S. Provisional Patent Application No. 62/704562, filed on May 15, 2020, entitled “HIGH DYNAMIC RANGE FAST CV SENSOR USING WIDE BANDGAP SILICON CARBIDE,” the contents of which are hereby incorporated by reference in its entirety.

### FIELD

The disclosure generally relates to methods and systems implementing a silicon carbide electrode in a cyclic voltammetry (CV) sensor.

### BACKGROUND

FSCV (fast scanning cyclic voltammetry) is a method that offers many advantages to sensing molecules like peptides, cytokines, and proteins. FSCV sweeps an electrode through stepped potentials at a fast rate, and if the electrical potential provides the level of energy required for a target chemical to experience redox reactions, the electron exchange can measured. Many redox reactions occur on the microsecond scale, so the microsecond scan rate of FSCV enables high resolution detection and quantification. Additionally, the potential delivered from the electrode facilitates the redox reaction for the target molecule, eliminating the requirement for surface functionalization with catalyst or linking molecules. This latter factor gives FSCV an advantage for chronic use as it does not stop functioning due to exhaustion of the functional molecular chemistry.

FSCV presents certain drawbacks which lower its final utility. A first major issue is that an electrical drift often develops during the constant cycling potential, making the removal of the background signal difficult and adding noise. This drift has been attributed to many different factors, some being temperature fluctuations, capacitive charging currents, non-specific species absorption, and alteration to the electrode material. Second, applying potentials greater than the limit of the electrode material generates the formation of surface oxide groups which increase the Faradic current of the electrode. Excessive potentials may lead to Faradaic currents which produce the hydrolysis of water, creating reactive oxygen and hydrogen species and contribute to the corrosion of the electrode itself. Biofouling, or the absorption of biomolecular elements to the surface of the electrode, will alter the impedance of the system, leading to a change in current. While these changes in Faradaic current can be stabilized through electrode conditioning, the addition of 15 minutes to 2 hours before signal acquisition can accurately occur limits the application of FSCV. Third, the target molecules demonstrate redox within the boundary potentials of −3V to +3V, a range that exceeds the water window limit for many materials. Finally, the fabrication methods for current sensors add issues of mass reproducibility, specificity, and physical fragility.

Carbon fibers are a mainstay FSCV electrode material. Carbon fibers, composed of graphitic sheets, offer a material platform that has demonstrated strong biocompatibility. While carbon demonstrates a good resistance to biofouling, bound oxygen located at the edges of the graphitic sheets facilitates absorption and increases reactions with target species leading to enhanced electron transfer. Carbon demonstrates good current stability within the potential range of −0.4 to +1.4V. However, carbon fibers present many difficulties. The fibers themselves are brittle and are easily broken. Variation in composition and size of the fibers create differences in electrical performance. Device fabrication and mass production can be different due to physical manipulation of the fibers. Carbon generated through the pyrolization of polymers or through the fabrication of graphene has become more prevalent, allowing modern interconnected circuit technology to assist in the fabrication of sensors. Finally, at potentials greater than +1V, oxidation along the edge of the graphitic sheets can produce CO₂ gas, corroding the electrode over time.

Transition metals allow the mass fabrication of devices with electrodes of controlled size and composition for increased specificity. These materials possess excellent ductility, making them less fragile than the brittle carbon electrodes. Their surfaces are catalytic in nature, facilitating electrochemical reactions as well as Faradaic electron transfer resulting in an amplification of signal and an increase in lower detection limit. However, the metal surfaces are susceptible to passivation, demonstrating a high degree of biofouling through protein absorption, a factor contributing to electrode drift. The materials generally are only stable at lower potentials, under +1.2V and above −0.6V, and contribute to hydrolysis if pushed beyond these limits. High currents also contribute to corrosion of the materials.

It is with respect to these and other considerations that the various aspects and embodiments of the present disclosure are presented.

### SUMMARY

An electrolytic voltammetry sensor comprises silicon carbide (SiC) uses the FSCV (fast scanning cyclic voltammetry) method for species sensing. The SiC may be single crystal SiC, and may be comprised within a SiC electrode. A system comprises a SiC electrode, and an applied voltage that is configured to apply voltage to the SiC electrode, wherein the voltage is swept within a range from a negative value to a positive value repeatedly at a rapid rate. The SiC electrode is configured to act as a biosensor in a FSCV process. The applied voltage is configured to be applied to the SiC electrode as a physiological species passes within a distance of the surface of the SiC electrode. The current resulting from redox electron exchange is received by the SiC electrode. A computing device may receive an output from the current, and use the output in a biomedical application. The biomedical application may be a COVID-based application. The applied potential range may be −2V to +2.8V.

In an implementation, a fast scan cyclic voltammetry (FSCV) electrochemical sensor comprising SiC is provided.

In an implementation, a system is provided that includes a SiC electrode, and an applied voltage that is configured to apply voltage to the SiC electrode, wherein the voltage is swept within a range from a negative value to a positive value repeatedly in a rapid fashion.

In an implementation, a method is provided that includes applying a voltage to a SiC electrode, wherein the voltage is swept within a range from a negative value to a positive value repeatedly; placing the SiC electrode near a physiological species while the voltage is being applied and swept over the range; sensing an output from the physiological species; and outputting the output.

This summary is provided to introduce a selection of concepts in a simplified form that are further described below in the detailed description. This summary is not intended to identify key features or essential features of the claimed subject matter, nor is it intended to be used to limit the scope of the claimed subject matter.

### BRIEF DESCRIPTION OF THE DRAWINGS

The accompanying drawings, which are in and constitute a part of this specification, illustrate certain examples of the present disclosure and together with the description, serve to explain, without limitation, the principles of the disclosure. Like numbers represent the same element(s) throughout the figures.

The foregoing summary, as well as the following detailed description of illustrative embodiments, is better understood when read in conjunction with the appended drawings. For the purpose of illustrating the embodiments, there is shown in the drawings example constructions of the embodiments; however, the embodiments are not limited to the specific methods and instrumentalities disclosed. In the drawings:

FIG. 1 is a diagram of an implementation of a fast scan cyclic voltammetry (CV) system that uses a silicon carbide (SiC) electrode;

FIG. 2 an operational flow of an implementation of a method for CV sensing using a SiC electrode;

FIG. 3 show charts of CV that compare standard (platinum) water window with 4H—SiC;

FIG. 4 is a photograph of a free-standing 16 channel SiC microelectrode array (MEA) prior to packaging;

FIG. 5 is a SEM micrograph detailing bonding pads for packaging a SiC MEA;

FIG. 6 is a SEM micrograph showing the sensor tip zone of an implementation of a SiC electrode; and

FIG. 7 shows an exemplary computing environment in which example embodiments and aspects may be implemented.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Unless defined otherwise, all technical and scientific terms used herein have the same meaning as commonly understood to one of ordinary skill in the art to which this invention belongs. As used in the specification and claims, the singular form “a,” “an,” and “the” include plural references unless the context clearly dictates otherwise. As used herein, the terms “can,” “may,” “optionally,” “can optionally,” and “may optionally” are used interchangeably and are meant to include cases in which the condition occurs as well as cases in which the condition does not occur. Publications cited herein are hereby specifically incorporated by reference in their entireties and at least for the material for which they are cited.

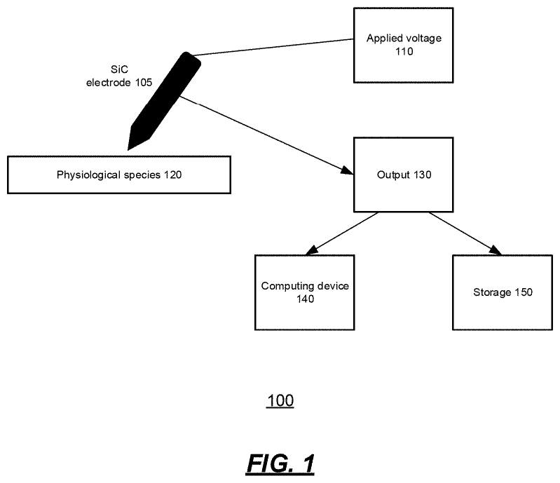

FIG. 1 is a diagram of an implementation of a cyclic voltammetry (CV) system 100 that uses a silicon carbide (SiC) electrode 105. Single crystal SiC, a semiconductor material, has demonstrated a stable capacitive electrochemical profile and can be fabricated into FSCV electrodes, such as the SiC electrode 105. SiC offers many advantages as compared to conventional FSCV electrodes. Silicon carbide addresses many of the problems associated with FSCV electrodes. The semiconducting material demonstrates a capacitive interaction with electrolytic environments through a depletion layer developing on the interacting surface creating a capacitive electrolytic interface, and demonstrates an extremely wide hydrolysis water window, which has been measured stable within in the range of −2.1V to +2.8V. SiC chemical inertness provides a lack of Faradaic surface interactions and facilitate the measurement of the redox currents which develop by the target molecules. The chemical resistance of the SiC material resists corrosion and the material has shown resistance to biofouling. It also has a multitude of fabrication methods, allowing wafer level production of devices. Additionally, it has demonstrated excellent compatibility within biological environments, which facilitates chronic sensor utilization.

An applied voltage 110 is applied in a rapid ramp between two potential limits to the SiC electrode 105. As a physiological species 120 passes within a distance of SiC electrode 105 the current output 130 is provided from the redox reactions from physiological species 120 to the SiC electrode 105 where it may be received, processed, analyzed, and/or displayed on a display of a computing device 140 and/or stored in storage 150 such as a database or computer memory, depending on the implementation.

The computing device 140 may be implemented using a variety of computing devices such as smartphones, desktop computers, laptop computers, and tablets. Other types of computing devices may be supported. A suitable computing device is illustrated in FIG. 7 as the computing device 700.

Electrochemical sensors require the measurement of current as a function of voltage whereby the voltage is swept between a certain range. Biosensors use the same mechanism in a process called CV. The applied voltage 110 provided to the SiC electrode 105 is swept within a certain range, from negative to positive values repeatedly, such as from −2V to +2.8V. The SiC electrode 105 acts as a biosensor in this CV process. The ability to sweep from −2V to +2.8V allows for a wide access to chemical species of interest in the physiological species 120. Such a high dynamic range is possible with an electrode comprising SiC, such as the SiC electrode 105. Conventional FSCV sensors use noble metals or carbon but have a much lower dynamic range. Electrochemical sensors require the measurement of current as a function of voltage whereby the voltage is swept from negative to positive values repeatedly. The dynamic range of sensors is dictated by the maximum voltage that the electrode can be swept to before non-linear processes occur. Conventional FSCV sensors, such as those made using platinum, are safely limited to less than ±1V, thus not allowing for the detection of numerous chemical species of interest. Other conventional sensors use C and can sweep −0.4 to +1.4 V.

The ability to sense numerous chemical and biological species is key to detecting contaminants, pathogens, viruses, as well as biological species, etc. The use of highly stable SiC, with 4H—SiC bandgap of 3.2 eV, as the electrode 105 allows for reliable and repeatable deep sensing of numerous physiological species 120 of interest. The SiC electrode 105 provided herein enables the full-range of sensing to be conducted.

In this manner, the SiC electrode 105, due to the compatibility of SiC with biological systems, opens up the possibility of using SiC for a plethora of biomedical applications. For example, a COVID-based application (e.g., a diagnosis application, a detection application, etc.) may be implemented using a certain electrochemical analyzer with high range sensing.

More particularly, an example application is the detection of various chemical and biological species in-vivo which is inherently a “wet” environment, highly amenable to electrochemical sensing. The present COVID-19 disease pandemic requires accurate sensing of either the virus itself or the presence of the disease in humans. The SiC electrode 105 may be comprised within an implantable-type sensing system, for example in the blood that provides accurate, real-time detection in humans.

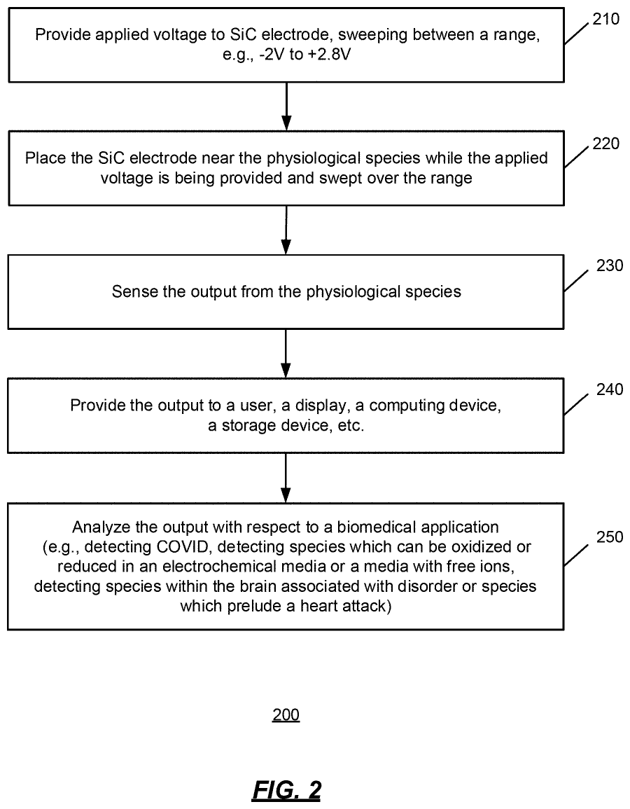

FIG. 2 an operational flow of an implementation of a method 200 for CV sensing using a SiC electrode, such as the SiC electrode 105.

At 210, an applied voltage 110 is provided to the SiC electrode 105 and swept from negative to positive values repeatedly, between a range of about −2V to about +2.8V. This range is not intended to be limiting, as any range can be used depending on the implementation.

At 220, the SiC electrode 105 is placed near the physiological species 120 while the applied 110 voltage is being provided and swept over the range.

At 230, the output 130 is sensed from the physiological species 120.

At 240, the output may be provided to a user, a display, a computing device, and/or to storage, etc., depending on the implementation.

At 250, the output may be analyzed with respect to a particular biomedical application, such as detecting COVID. In some implementations, the sensor may not sense COVID directly. The sensor may detect certain proteins from COVID if they can experience redox reactions. The sensor has a wider sensing capability. The sensor can detect species within the brain associated with disorder or species which prelude a heart attack.

The sensors are made to detect species which can be oxidized or reduced in an electrochemical media, or a media with free ions. The sensor can detect different species in the brain and the heart, and possibly certain proteins.

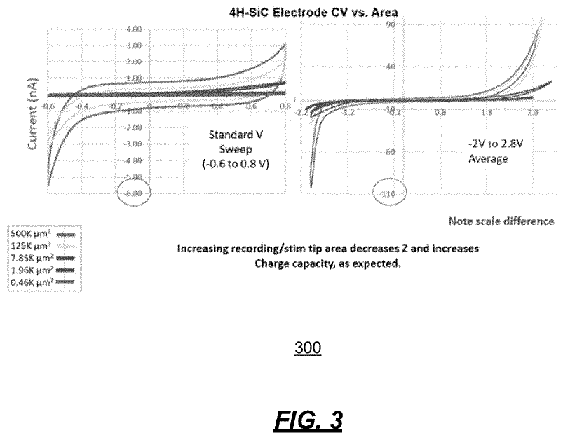

FIG. 3 show charts 300 of CV that compare standard (platinum) water window with 4H—SiC. A 4H—SiC electrode was fabricated and its CV characteristics measured. The left chart of FIG. 3 shows the CV profile from −0.6 to +0.8 V which is the standard water window width for platinum electrodes. The right chart of FIG. 3 shows the true 4H—SiC water window where the CV profile was measured from −2.2 to +3V. This data demonstrates the significant advantage to using SiC for FSCV sensing as numerous chemical/biological species are not accessible with the more narrow water window. As is known, in then ranges from −2 to −1V and from +1 to +2 V, many different chemical/biological species can be detected. Thus, the SiC electrodes provided herein can be used as FSCV sensors to detect more elements/items.

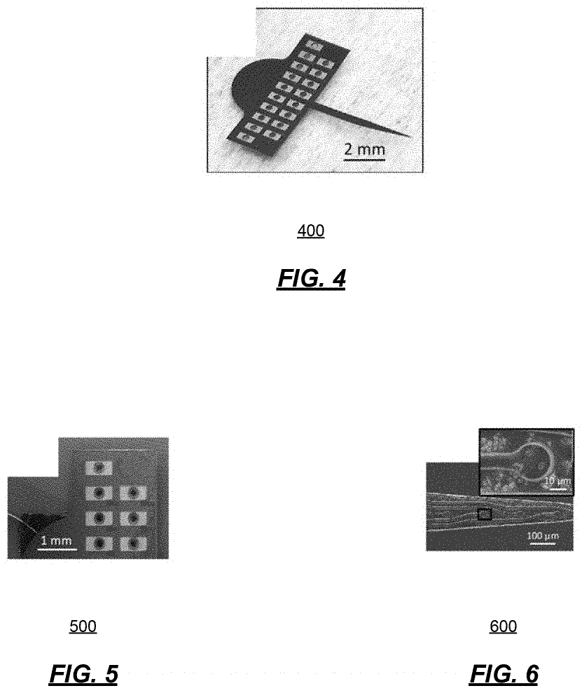

FIG. 4 is a photograph 400 of a free-standing 16 channel SiC microelectrode array (MEA) prior to packaging. In this device fabricated from SiC, SiC is used as the body of the implant, SiC is used for the electrode, and SiC is used for the electrical traces and electrical isolation.

FIG. 5 is a SEM micrograph 500 detailing bonding pads for packaging a SiC MEA, such as the Si MEA of FIG. 4. Gold bond pads allow it to be soldered to a connector that can enable connection to potentiostat/galvanostat electronics for and computer for electrochemical evaluation.

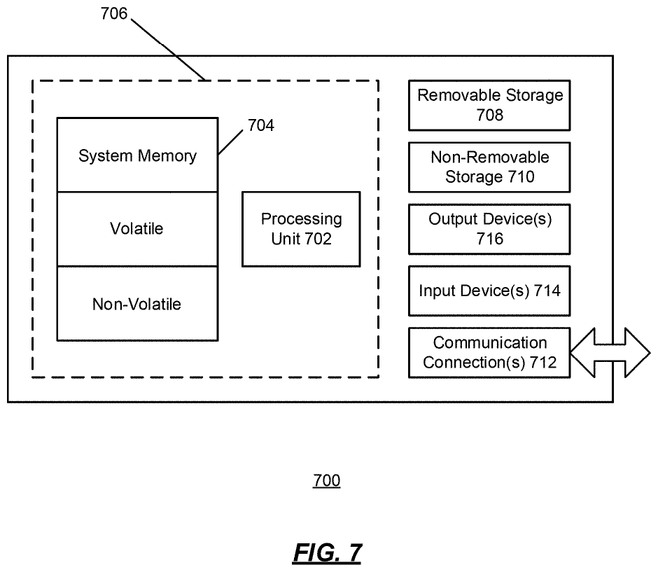

FIG. 6 is a SEM micrograph 600 showing the sensor tip zone of an implementation of a SiC electrode. The shank is 5.1 mm long (2.4 mm tapered portion). The tab is 6.64 mm wide and 2.3 mm long.

FIG. 7 shows an exemplary computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 7, an exemplary system for implementing aspects described herein includes a computing device, such as computing device 700. In its most basic configuration, computing device 700 typically includes at least one processing unit 702 and memory 704. Depending on the exact configuration and type of computing device, memory 704 may be volatile (such as random access memory (RAM)), non-volatile (such as read-only memory (ROM), flash memory, etc.), or some combination of the two. This most basic configuration is illustrated in FIG. 7 by dashed line 706.

Computing device 700 may have additional features/functionality. For example, computing device 700 may include additional storage (removable and/or non-removable) including, but not limited to, magnetic or optical disks or tape. Such additional storage is illustrated in FIG. 7 by removable storage 708 and non-removable storage 710.

Computing device 700 typically includes a variety of computer readable media. Computer readable media can be any available media that can be accessed by the device 700 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 704, removable storage 708, and non-removable storage 710 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 700. Any such computer storage media may be part of computing device 700.

Computing device 700 may contain communication connection(s) 712 that allow the device to communicate with other devices. Computing device 700 may also have input device(s) 714 such as a keyboard, mouse, pen, voice input device, touch input device, etc. Output device(s) 716 such as a display, speakers, printer, etc. may also be included. All these devices are well known in the art and need not be discussed at length here.

In an implementation, a fast scan cyclic voltammetry (FSCV) electrochemical sensor comprising silicon carbide (SiC) is provided.

Implementations may include some or all of the following features. The SiC is single crystal SiC. The SiC is comprised within a SiC electrode. The SiC electrode is configured to act as a biosensor in a FSCV process. The SiC electrode is comprised within a electrochemical-type voltammetry sensing system to provide accurate, real-time detection in humans. The SiC electrode is comprised within a sensing system to detect a molecules which experience redox reactions in-vitro or in-vivo to diagnose or detect the onset of disease.

In an implementation, a system is provided that includes a silicon carbide (SiC) electrode, and an applied voltage that is configured to apply voltage to the SiC electrode, wherein the voltage is swept within a range from a negative value to a positive value repeatedly in a rapid fashion.

Implementations may include some or all of the following features. The SiC electrode is a single crystal SiC. The SiC electrode is configured to act as a biosensor in a fast scan cyclic voltammetry (FSCV) process. The applied voltage is configured to be applied to the SiC electrode as it passes within a distance of a physiological species. The system further comprises a computing device that is configured to receive an output from the physiological species. The computing device is configured to use the output in a biomedical application. The biomedical application is detecting species which can be oxidized or reduced in an electrochemical media or a media with free ions. The range is −2V to +2.8V.

In an implementation, a method is provided that includes applying a voltage to a silicon carbide (SiC) electrode, wherein the voltage is swept within a range from a negative value to a positive value repeatedly; placing the SiC electrode near a physiological species while the voltage is being applied and swept over the range; sensing an output from the physiological species; and outputting the output.

Implementations may include some or all of the following features. The method further comprises analyzing the output with respect to a biomedical application. The method further comprises detecting species which can be oxidized or reduced in an electrochemical media or a media with free ions. Outputting the output comprises providing the output to at least one of a user, a display, a computing device, or a storage device. The SiC electrode comprises single crystal SiC. The range is −2V to +2.8V.

It should be understood that while the present disclosure has been provided in detail with respect to certain illustrative and specific aspects thereof, it should not be considered limited to such, as numerous modifications are possible without departing from the broad spirit and scope of the present disclosure as defined in the appended claims. It is, therefore, intended that the appended claims cover all such equivalent variations as fall within the true spirit and scope of the invention.

What is claimed is:

1. A fast scan cyclic voltammetry (FSCV) electrochemical sensor comprising silicon carbide (SiC).

2. The FSCV sensor of claim 1, wherein the SiC is single crystal SiC.

3. The FSCV sensor of claim 1, wherein the SiC is comprised within a SiC electrode.

4. The FSCV sensor of claim 3, wherein the SiC electrode is configured to act as a biosensor in a FSCV process.

5. The FSCV sensor of claim 3, wherein the SiC electrode is comprised within a electrochemical-type voltammetry sensing system to provide accurate, real-time detection in humans.

6. The FSCV sensor of claim 3, wherein the SiC electrode is comprised within a sensing system to detect a molecules which experience redox reactions in-vitro or in-vivo to diagnose or detect the onset of disease.

7. A system comprising:
a silicon carbide (SiC) electrode; and
an applied voltage that is configured to apply voltage to the SiC electrode, wherein the voltage is swept within a range from a negative value to a positive value repeatedly in a rapid fashion.

8. The system of claim 7, wherein the SiC electrode is a single crystal SiC.

9. The system of claim 7, wherein the SiC electrode is configured to act as a biosensor in a fast scan cyclic voltammetry (FSCV) process.

10. The system of claim 7, wherein the applied voltage is configured to be applied to the SiC electrode as it passes within a distance of a physiological species.

11. The system of claim 7, further comprising a computing device that is configured to receive an output from the physiological species.

12. The system of claim 11, wherein the computing device is configured to use the output in a biomedical application.

13. The system of claim 12, wherein the biomedical application is detecting species which can be oxidized or reduced in an electrochemical media or a media with free ions.

14. The system of claim 7, wherein the range is −2V to +2.8V.

15. A method comprising:
applying a voltage to a silicon carbide (SiC) electrode, wherein the voltage is swept within a range from a negative value to a positive value repeatedly;
placing the SiC electrode near a physiological species while the voltage is being applied and swept over the range;
sensing an output from the physiological species; and
outputting the output.

16. The method of claim 15, further comprising analyzing the output with respect to a biomedical application.

17. The method of claim 16, further comprising detecting species which can be oxidized or reduced in an electrochemical media or a media with free ions.

18. The method of claim 15, wherein outputting the output comprises providing the output to at least one of a user, a display, a computing device, or a storage device.

19. The method of claim 15, wherein the SiC electrode comprises single crystal SiC.

20. The method of claim 15, wherein the range is −2V to +2.8V.
